(19) United States
(12) Patent Application Publication
(10) Pub. No.: US 2022/0181028 A1
(43) Pub. Date: Jun. 9, 2022

(54) ALGORITHM FOR THE IDENTIFICATION AND PHENOTYPING OF NONALCOHOLIC FATTY LIVER DISEASE PATIENTS

(71) Applicant: THE TRUSTEES OF COLUMBIA UNIVERSITY IN THE CITY OF NEW YORK, New York, NY (US)

(72) Inventors: Anna BASILE, Flushing, NY (US); Julia WATTACHERIL, New York, NY (US); Nicholas TATONETTI, New York, NY (US)

(73) Assignee: THE TRUSTEES OF COLUMBIA UNIVERSITY IN THE CITY OF NEW YORK, New York, NY (US)

(21) Appl. No.: 17/679,707

(22) Filed: Feb. 24, 2022

Related U.S. Application Data
(63) Continuation of application No. PCT/US2020/047947, filed on Aug. 26, 2020.
(60) Provisional application No. 62/891,748, filed on Aug. 26, 2019.

(51) Int. Cl. G16H 50/20; G16H 10/60; G16H 50/30

(52) U.S. Cl. CPC G16H 50/20; G16H 50/30; G16H 10/60

(57) ABSTRACT

System and methods for diagnosing nonalcoholic fatty liver disease (NAFLD)/nonalcoholic steatohepatitis (NASH) in patients are disclosed. The system can comprise one or more processors and one or more computer-readable non-transitory storage media coupled to the one or more of processors including instructions operable when executed by one or more of the processor. The system can be configured to select at least one patient with a risk indicator using an electronic health record (EHR) database, determine that the at least one patient fails to meet exclusion criteria, and display the at least one patient in response to the determination. The risk indicator can be associated with NAFLD and/or NASH. Methods for diagnosing NAFLD/NASH in patients are disclosed are also provided.

### CROSS-REFERENCE TO RELATED APPLICATION

This application is a continuation of International Patent Application No. PCT/US 2020/047947 filed Aug. 26, 2020, which claims priority to U.S. Provisional Application No. 62/891,748, which was filed on Aug. 26, 2019, the entire contents of which are incorporated by reference herein.

### BACKGROUND

Nonalcoholic fatty acid liver disease (NAFLD) can be a cause of chronic liver disease which can affect between 80 and 100 million individuals in the United States. This disease can be benign, aggressive, or harmful from a liver perspective and can be associated with cardiometabolic outcomes. In a nonalcoholic fatty liver, excess fat can accumulate in the liver cells. Such build up of fat in the liver can induce inflammation and damage to the liver resulting in non-alcoholic steatohepatitis (NASH). NAFLD and NASH can lead to cirrhosis, hepatocellular carcinoma and become indications for liver transplantation in adults and children. Currently, no approved pharmacologic treatment for NASH is available.

Certain existing methods can require multiple clinical tests to screen NAFLD/NASH patients. Furthermore, while certain tests can be ordered by liver specialists, the burden of the disease is not necessarily placed under the care of liver specialists. Accordingly, there remains a need for improved techniques that can identify patients at risk for NAFLD and NASH from data that can be readily and routinely acquired from patients to facilitate access to appropriate care.

### SUMMARY

The disclosed subject matter provides systems and methods for identifying nonalcoholic fatty liver disease (NAFLD) and nonalcoholic steatohepatitis (NASH) in patients using clinical data available in the electronic health record. An example system can include one or more processors and one or more computer-readable non-transitory storage media coupled to one or more of the processors. The storage media can store instructions to cause the system to select at least one patient with a risk indicator using an electronic health record (EHR) database, determine that the at least one patient fails to meet exclusion criteria, and display the at least one patient in response to the determination. In example embodiments, the disclosed risk factor can be associated with NAFLD and/or NASH. The risk factor can include demographic data (e.g., age, sex, etc.), diagnosis codes, procedure codes, laboratory measurements, medication history, pathology codes, radiology codes, or combinations thereof. For example, the risk factor can include patient data related to type 2 diabetes, obesity, abnormal liver enzymes, hyperlipidemia, hypertension, chronic nonalcoholic liver disease, nonalcoholic steatohepatitis, steatosis, cirrhosis, and combinations thereof.

In certain embodiments, the disclosed system can assess exclusion criteria for screening patients. The exclusion criteria can include demographic data, diagnosis codes, procedure codes, laboratory measurements, medication history, pathology codes, radiology codes, or combinations thereof. For example, the exclusion criteria can include patient data related to alcohol use/abuse, type 1 diabetes, viral hepatitis infection, HIV infection, age, or combinations thereof.

In certain embodiments, the disclosed system can be configured to verify hepatic steatosis of the at least one patient using a radiology report and/or a pathology report. In some embodiments, the disclosed radiology report can include an ultrasound report, a CT scan report, a MRI report, or combinations thereof.

In certain embodiments, the disclosed system can be further configured to determine that the patient receives a weight-loss surgery. The disclosed weight-loss surgery can include a laparoscopy procedure, a gastric restrictive procedure, a bariatric procedure, a bariatric revision, or combinations thereof.

In certain embodiments, the disclosed system can be further configured to determine that the at least one patient has an end-stage liver-related outcome. The end-stage liver related outcome can include portal hypertension, hepatorenal syndrome, primary bacterial peritonitis, ascites, complications of transplanted liver, hepatic encephalopathy, cirrhosis, hepatocellular carcinoma, hepatopulmonary syndrome, hepatic failure, esophageal varices, esophagogastroduodenoscopy or combinations thereof.

In certain embodiments, the disclosed system can perform a quality control by excluding a patient who has less than two risk factors or less than three occurrences of the risk factors.

In certain embodiments, an example method for diagnosing NAFLD/NASH patients can include selecting at least one patient with a risk indicator using an EHR database, determining that the at least one patient fails to meet exclusion criteria, and displaying the at least one patient in response to the determination. The risk indicator can be associated with NAFLD and/or NASH. In some embodiments, the example method can further include verifying hepatic steatosis of the at least one patient using a radiology report and/or a pathology report. In some embodiments, the example method can further include performing a quality control by excluding a patient who has less than two risk indicators or less than three occurrences of the risk indicator. In certain embodiments, the example method can further include determining that the at least one patient receives a weight-loss surgery. In some embodiments, the example method can further include determining that the at least one patient has an end-stage liver-related outcome.

### BRIEF DESCRIPTION OF THE DRAWINGS

Further features and advantages of the present disclosure will become apparent from the following detailed description taken in conjunction with the accompanying figures showing illustrative embodiments of the present disclosure, in which:

FIG. 1 is a flow diagram illustrating an example process in accordance with the present disclosure.

FIG. 2 is an exemplary workflow of the disclosed system in accordance with the present disclosure.

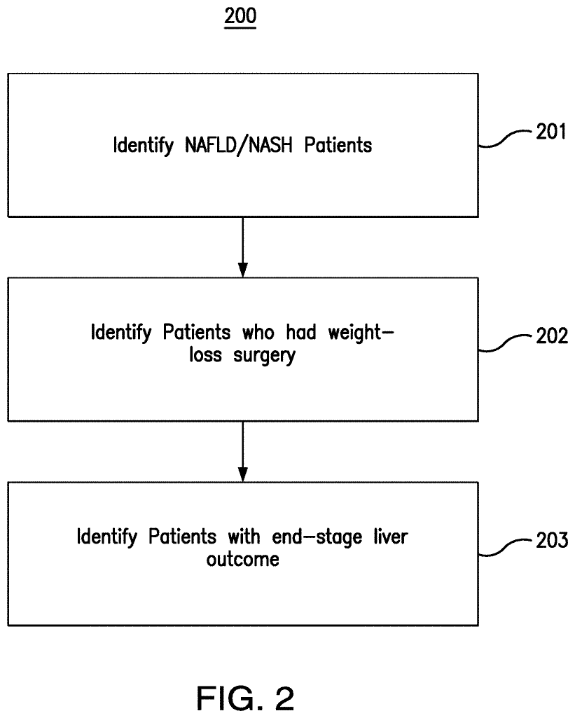

FIG. 3 is a diagram illustrating example performance to identify NAFLD/NASH patients in accordance with the disclosed subject matter.

FIG. 4 is a diagram illustrating example performance to identify patients who received weight-loss surgery in accordance with the disclosed subject matter.

FIG. 5 is a diagram illustrating example performance to identify patients with end-stage liver outcome in accordance with the disclosed subject matter.

Throughout the figures, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments.

### DETAILED DESCRIPTION

The disclosed subject matter provides techniques for diagnosing nonalcoholic fatty liver disease (NAFLD) and nonalcoholic steatohepatitis (NASH) in patients. The disclosed subject matter can assess various data that can be readily and routinely acquired from patients for predicting risks of NAFLD and NASH, thereby tailoring need for additional clinical testing in certain risk populations.

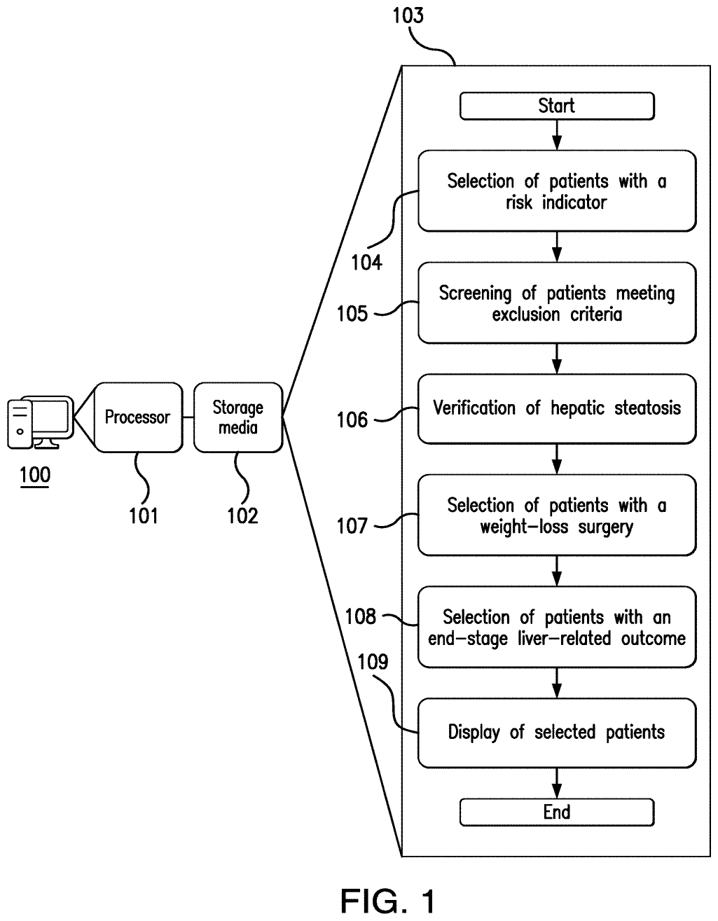

As shown FIG. 1, an exemplary system 100 can include one or more processors 101 and one or more computer-readable non-transitory storage media 102 coupled thereto. For example, the processor 101 can be an electronic circuitry (e.g., central processing unit, graphics processing unit, digital signal processor, etc.) within a computer/server 100 that can include a non-transitory storage media 102. Instructions 103 can include a set of machine language that a processor can understand and execute. As shown in FIG. 1, the disclosed media 102 can include instructions 103 operable when executed by one or more of the processors 101 to cause the system 100 to perform various operations and analyses 104-109 for diagnosing NAFLD and NASH in patients.

In certain embodiments, the disclosed system can be configured to select at least one patient with a risk indicator 104. The risk indicator can be associated with a target disease or symptom. The target disease/symptom associated indicator can include a diagnosis code, a procedure code, a laboratory measurement, a medication history, a pathology code, a radiology code, demographic data and combinations thereof. For example, certain risk indicators can be associated with NAFLD and/or NASH. The NAFLD/NASH associated risk indicators can include patient data related to type 2 diabetes (e.g., hemoglobin A1C≥5.7), obesity (e.g., body mass index≥30), abnormal liver enzymes (e.g., alanine aminotransferase≥40), hyperlipidemia (e.g., total cholesterol≥200 or low-density lipoproteins≥130), hypertension, chronic nonalcoholic liver diseases, nonalcoholic steatohepatitis, steatosis, cirrhosis, or combinations thereof.

In certain embodiments, the disclosed system can be configured to select the at least one patient using a database. The database can be a public or a private. For example, an exemplary system can obtain patient data (e.g., risk indicators) from an electronic health record (EHR) database. In some embodiments, the database can be private. The private database can include protected health information, and cannot publicly available. In some embodiments, the disclosed database can be obtained from any medical centers, institutions, and/or hospitals.

In certain embodiments, the disclosed system can be configured to identify patients who meet exclusion criteria 105. The exclusion criteria can include a diagnosis code, a procedure code, a laboratory measurement, a medication history, a pathology code, a radiology code, demographic data and combinations thereof. For example, certain exclusion criteria can include patient data related to alcohol abuse, type 1 diabetes, viral hepatitis infection, HIV infection, age (e.g., ≤18), or combinations thereof In some embodiments, the disclosed system can be configured to deselect/remove the patients who meet the exclusion criteria from the selected patients with the risk indicator 105.

In certain embodiments, the disclosed system can be configured to verify hepatic steatosis of the selected patients 106. Hepatic steatosis can be verified by histologic description based on pathologist review of liver biopsies contained within clinical reports or imaging modalities that incorporate signal detection that has been associated with the presence of intrahepatic fat. For example, increased echogenicity within an abdominal ultrasound report (with appropriate exclusion criteria) can be correlated with intrahepatic fat. In some embodiments, the verification process can be performed using a radiology report and/or a pathology report. For example, the radiology report can include an ultrasound report, a CT scan report, a MRI report, or combinations thereof. The pathology report can include reports obtained via liver biopsy for NASH, NAFLD, steatosis, steatohepatitis, fatty liver, or cirrhosis.

In certain embodiments, the disclosed system can be configured to perform a quality control process by excluding a patient who has less than two risk factors or less than three occurrences of a single risk indicator. Certain electronic health records can include errors that can range from data entry errors to incorrect code usage. To reduce the chance errors and the false positive rate, the process can require patients to have at least two distinct risk factors (e.g. a diagnosis of hypertension and a diagnosis of obesity) or three occurrences of a single risk indicator (i.e. the patient was diagnosed with a risk indicator on 3 different medical visits).

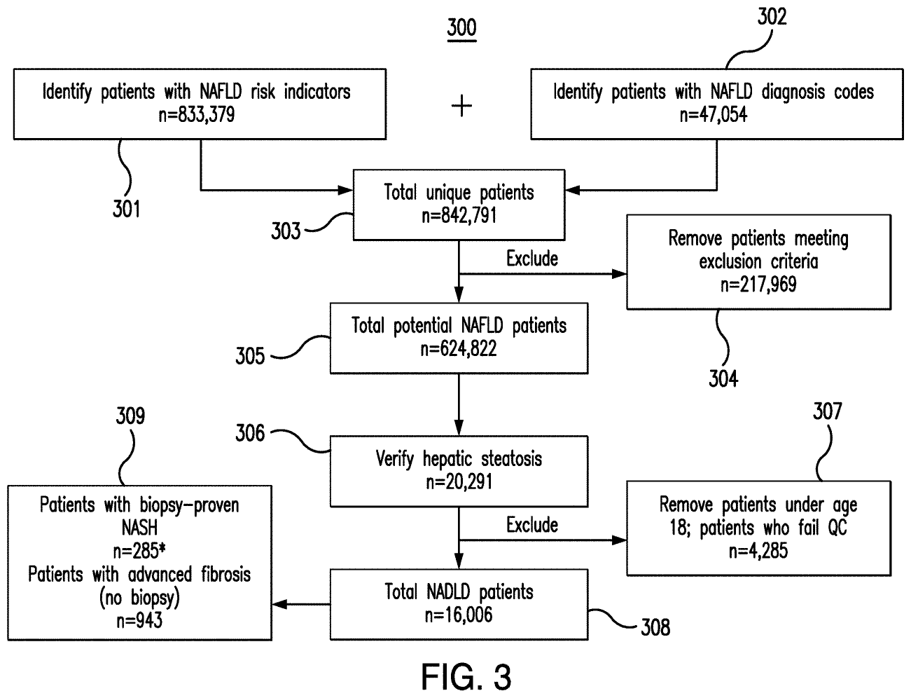

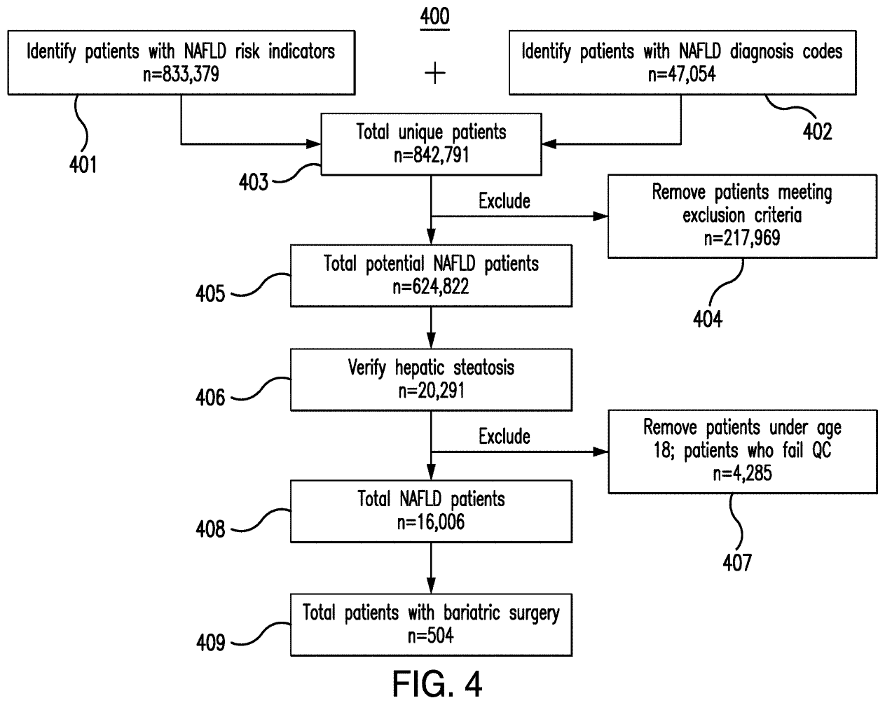

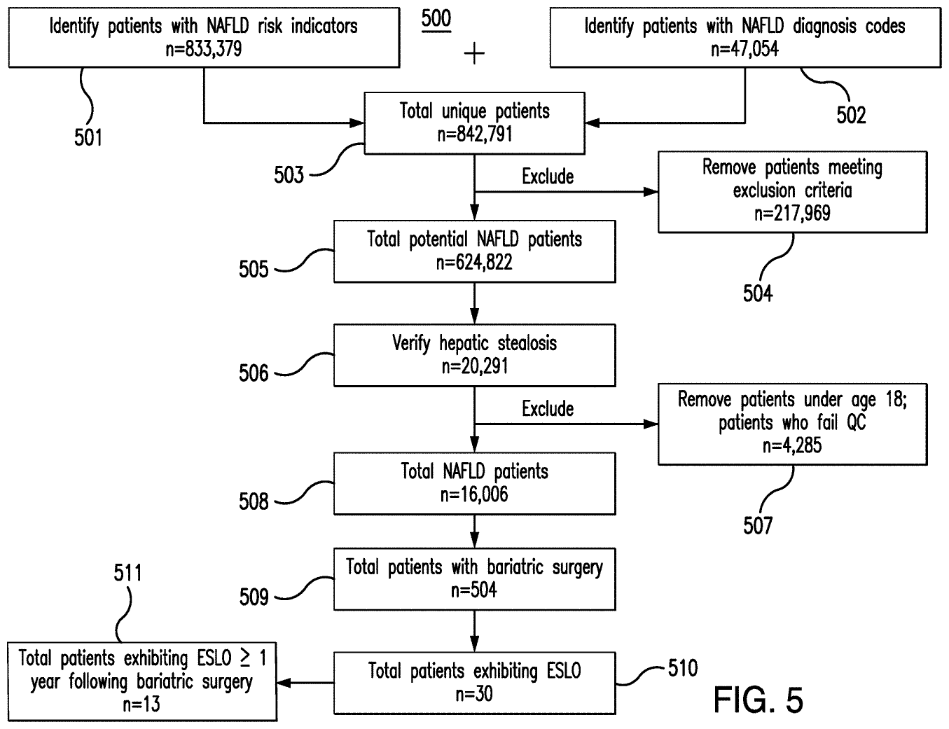

In certain embodiments, the disclosed system can be configured to identify patients with a weight-loss surgery 107. The identification of patients with a weight-loss surgery can be performed independently from portions of the method, and can be a continuation of an example illustrated in FIG. 3. As an example, to improve the accuracy of the diagnosis, the disclosed system can further identify patients who receive a weight-loss surgery 202 from selecting the selected patients with the NAFLD/NASH associated risk indicators 201. The weight-loss surgery can include a laparoscopy procedure, a gastric restrictive procedure, a bariatric procedure, a bariatric revision, or combinations thereof. For example, as shown in FIG. 3, total patients (e.g., more than 800, 000) with NAFLD risk indicators 301 or diagnosis codes 302 can be identified from electronic health record databases 303. Total potential NAFLD patients 305 can be obtained by removing patients who meet exclusion criteria 304 from total patients with NAFLD indicators/diagnosis codes 303. The potential NAFLD patients can be further assessed for verifying hepatic steatosis. Total NAFLD patients 308 can be obtained by removing patients who meet the second exclusion criteria and/or fail to pass the quality control 307. Among the NAFLD patients, patients with biopsy-proven NASH and/or advanced fibrosis can be further identified 309. As shown in FIG. 4, among the NAFLD patients, patients who have had bariatric surgery can be further identified. In certain embodiments, patients who continue to exhibit liver-related outcomes following weight-loss surgery can be also identified (FIG. 5).

In certain embodiments, the disclosed system can be configured to identify patients with an end-stage liver outcome 108. The end-stage liver outcome can include patient date related to Model for End Stage Liver Disease (MELD) score, portal hypertension, hepatorenal syndrome, primary bacterial peritonitis, ascites, complications of transplanted liver, hepatic encephalopathy, cirrhosis, hepatopulmonary syndrome, hepatic failure, esophageal varices, esophagogastroduodenoscopy, or combinations thereof. The identification of patients with an end-stage liver outcome 108 can be performed independently from other portions of the method, and can be a continuation of an example illustrated in FIG. 4. For example, as shown in FIG. 5, patients exhibiting the end-stage liver outcome can be further identified 510. In some embodiments, patients exhibiting an end-stage liver disease outcome after bariatric surgery can be identified 511. These outcomes identified by diagnostic codes and can be subjected to clinical verification.

In certain embodiments, the MELD score can be calculated to stratify patients by expected mortality and to decompensate liver disease with regards to liver transplantation. The formula for calculating a MELD score can be:

10*((0.957*ln(Creatinine))+(0.378*ln(Bilirubin))+(1.12*ln(INR)))+6.43 (1)

For the calculation, laboratory measurements (e.g., creatinine, Bilirubin, and INR) taken at least one year following weight-loss surgery for each patient can be extracted. The measurements (e.g., creatinine, Bilirubin, and INR) can be taken within 30-days of each other, and the max value for each measurement type can be selected. MELD scores can be then calculated per patient using this information. Table 1 below lists the measurement codes used for the MELD score calculation.

TABLE 1
Measurements for the MELD score calculation

| OMOP Concept ID | OMOP Concept Name | LOINC code |
|---|---|---|
| 3022217 | INR | 6301-6 |
| 3032080 | INR in Blood by Coagulation Assay | 34714-6 |
| 3024128 | Total Bilirubin | 1975-2 |
| 3016723 | Creatinine serum/plasma | 2160-0 |

In certain embodiments, the disclosed system can be further configured to identify patients with advanced fibrosis. For example, a non-biopsied patient group can be scored using Fibrosis-4 (FIB-4), AST to Platelet Ratio Index (APRI), and NAFLD Fibrosis Score (NAFLD-FS) calculations to discern patients with advanced fibrosis. FIB-4, APRI, and FS can be obtained using the following metrics:

$$\begin{matrix}Fib - 4 = \frac{Age ( years ) * AST Level ( \frac{U}{L} )}{Platelet Count ( \frac{{10}^{9}}{L} ) * \sqrt{ALT} ( \frac{U}{L} )} & ( 2 ) \\ APRI = \frac{{(}^{AST Level \frac{IU}{L}} AST ( Upper Limit of Normal ) ( \frac{IU}{L} ) )}{Platelet Count ( {10}^{9} L )} * 100 & ( 3 ) \\ NAFLD - FS = - 1.675 + 0.037 * age ( years ) + 0.094 * BMI ( \frac{kg}{m 2} ) + 1.13 * \frac{IFG}{diabetes ( yes = 1 , no = 2 )} + 0.99 * \frac{AST}{ALT} ratio - 0.013 * platelet count ( \frac{{10}^{9}}{L} ) - 0.66 * albumin ( \frac{g}{dL} ) & ( 4 )\end{matrix}$$

These noninvasive scoring techniques have been applied to chronic liver disease, including NAFLD, to assist with the determination of degrees of fibrosis based on commonly available clinical data.

### EXAMPLE

The presently disclosed subject matter will be better understood by reference to the following Example. The Example provided as merely illustrative of the disclosed methods and systems, and should not be considered as a limitation in any way.

Among other features, the example illustrates the identification of patients with NAFLD and NASH within large electronic health record (EHR) databases for targeted intervention based on clinically relevant phenotypes.

This example considered the rapid identification of patients with NAFLD and NASH using EHRs from 6.4 million adult patients. Structured medical record data (diagnoses, medications, procedures, and demographics) were standardized by mapping to the Observational Medical Outcomes Partnership (OMOP) common data model and stored in MySQL. The example was semi-automated, guided by clinical validation and involved selecting patients with NAFLD risk indicators, removing patients meeting exclusion criteria, and machine confirmation of language indicators of hepatic steatosis. SQL queries were made on the structured data as follows.

First, NAFLD patients were identified using two criteria: presence of a NAFLD risk indicator or presence of a NAFLD diagnosis code. Patients only needed to be diagnosed with 1 risk indicator or NAFLD diagnosis code for cohort inclusion. NAFLD risk indicators include diagnosis of the following: type 2 diabetes (Table 2), obesity (Table 3), abnormal liver enzymes (Table 4), hyperlipidemia (Table 5), or hypertension (Table 6). Diagnosis codes used by the algorithm along with selection criteria for the NAFLD risk indicators are listed in Tables 2-6. Each table lists the OMOP name and code id along with the specific diagnostic code and code type. Criteria for inclusion for ICD 9/10 diagnoses was 1 diagnosis (dx). Laboratory measures (code type=LOINC) can list appropriate cutoffs for cohort inclusion. 833,379 patients with NAFLD risk indicators were identified. The NAFLD diagnosis codes used for patient selection are listed in Table 7. For the ICD 9/ICD 10 codes, patients with 1 diagnosis of the specified code were included in the cohort. For laboratory measurements (LOINC code), cutoff values for cohort inclusion are listed in these tables. 47,054 patients were identified with NAFLD diagnosis codes. 842,791 total unique patients were identified.

TABLE 2
Type 2 diabetes

| OMOP | | | | |
|---|---|---|---|---|
| Concept | OMOP Concept | | | Criteria for |
| ID | Name | Code Type | Specific Code | Inclusion |
| 201826 | Type 2 diabetes | ICD 9/ICD 10 | I9:250.00, I9:250.02, I10:E11.00, I10:E11.630 | 1 dx |
| | mellitus | | | |
| 4193704 | Type 2 diabetes | ICD 9/ICD 10 | I10:E11.9 | 1 dx |
| | mellitus without | | | |
| | complication | | | |
| 40482801 | Type II diabetes | ICD 9/ICD 10 | I9:250.02 | 1 dx |
| | mellitus uncontrolled | | | |
| 376065 | Neurologic disorder | ICD 9/ICD 10 | E11.49 | 1 dx |
| | associated with type 2 | | | |
| | diabetes mellitus | | | |
| 4044391 | Diabetic neuropathy | ICD 9/ICD 10 | I10:E13.40, I10:E08.40 | 1 dx |
| 376979 | Diabetic cataract | ICD 9/ICD 10 | I9:366.41, I10:E08.36 | 1 dx |
| 4009303 | Diabetic ketoacidosis | ICD 9/ICD 10 | E10.10, I10:E13.10, I10:E09.10, I10:E08.10 | 1 dx |
| | without coma | | | |
| 4159742 | Diabetic foot ulcer | ICD 9/ICD 10 | E08.621, I10:E13.621, I10:E08.621 | 1 dx |
| 37018196 | Prediabetes | ICD 9/ICD 10 | I10:R73.03 | 1 dx |
| 192279 | Diabetic renal disease | ICD 9/ICD 10 | I9:250.4, I10:E13.22, I10:E13.29, E13.21, | 1 dx |
| | | | I10:E09.21, I10:E08.22, I9:249.41, I10:E08.21, | |
| | | | I9:249.40, I10:E13.21, I10:E09.22, I10:E08.29, | |
| | | | I10:E09.29 | |
| 195771 | Secondary diabetes | ICD 9/ICD 10 | I9:249.00, E08.22, I9:249.01, I9:249.80, | 1 dx |
| | mellitus | | E08.29, I9:249.61, 249.40, E08.21, I9:249.90, | |
| | | | I9:249.41, I9:249.51, I9:249.60, I9:249.40, | |
| | | | I9:249.20, I9:249.21, I9:249.81, I9:249.50, | |
| | | | E08.630, I9:249.70, I9:249.10, I9:249.11, | |
| | | | I10:E08.36, I9:249.91, I9:249.30, E08.618, | |
| | | | I9:249.71 | |
| 201820 | Diabetes mellitus | ICD 9/ICD 10 | I9:250, I10:E13.65, I10:E13.00, 250, E13.649, | 1 dx |
| | | | I10:E08.00, E08.620 | |
| 321822 | Peripheral circulatory | ICD 9/ICD 10 | I10:E13.59, 250.7, E08.59, I9:249.70, E13.51, | 1 dx |
| | disorder associated | | E08.52, I9:249.71, I10:E08.51 | |
| | with diabetes mellitus | | | |
| 376112 | Diabetic | ICD 9/ICD 10 | I9:357.2, I10:E13.42, I10:E08.42 | 1 dx |
| | polyneuropathy | | | |
| 377552 | Moderate | ICD 9/ICD 10 | 362.05, I9:362.05, E08.331 | 1 dx |
| | nonproliferative | | | |
| | diabetic retinopathy | | | |
| 380096 | Proliferative diabetic | ICD 9/ICD 10 | I9:362.02, E08.359, I10:E08.3553, | 1 dx |
| | retinopathy | | I10:E08.351, I10:E13.359, I10:E13.3592, | |
| | | | I10:E13.3593 | |
| 380688 | Hypoglycemic coma | ICD 9/ICD 10 | I9:251.0, 249.31, I9:249.30 | 1 dx |
| 436940 | Metabolic syndrome X | ICD 9/ICD 10 | E88.81, I9:277.7, I10:E88.81 | 1 dx |
| 442793 | Diabetic complication | ICD 9/ICD 10 | I9:250.9, 249.91, E13.8, I10:E08.8, I9:249.80, | 1 dx |
| | | | E13.628, I10:E08.69, 249.81, I9:249.90, | |
| | | | I10:E13.8, E13.618, I10:E08.59, E08.638, | |
| | | | I9:249.81, I10:E08.630, I9:249.91, E08.628, | |
| | | | I10:E13.69, I10:E13.638, I10:E09.8, 249.9, | |
| | | | I10:E09.69 | |
| 443727 | Diabetic ketoacidosis | ICD 9/ICD 10 | I9:250.1, 249.10, I9:249.10, I9:249.11 | 1 dx |
| 443729 | Peripheral circulatory | ICD 9/ICD 10 | I9:250.70, E11.59, I9:250.72, 250.70, | 1 dx |
| | disorder associated | | I10:E11.59, I10:E11.51 | |
| | with type 2 diabetes | | | |
| | mellitus | | | |
| 443730 | Neurologic disorder | ICD 9/ICD 10 | E08.49, 250.6, E13.49, I9:249.61, 249.60, | 1 dx |
| | associated with | | I10:E13.49, I9:249.60, I9:250.6, I10:E08.49, | |
| | diabetes mellitus | | E09.42 | |
| 443732 | Disorder due to type 2 | ICD 9/ICD 10 | 250.90, 110:E11.8, I9:250.90, I9:250.80, | 1 dx |
| | diabetes mellitus | | I9:250.92, I9:250.82, 250.80, E11.69, E11.628, | |
| | | | I10:E11.69, E11.620, I10:E11.638 | |
| 443733 | Diabetic oculopathy | ICD 9/ICD 10 | I9:250.52, I10:E11.39, 250.52, E11.359, | 1 dx |
| | associated with type 2 | | I10:E11.319 | |
| | diabetes mellitus | | | |
| 443767 | Diabetic oculopathy | ICD 9/ICD 10 | I9:250.50, E13.311, E13.36, I9:249.51, E13.39, | 1 dx |
| | | | I9:250.5, I9:249.50, I10:E13.39, E08.39 | |
| 444369 | Hyperosmolality | ICD 9/ICD 10 | 249.21, I9:249.20 | 1 dx |
| 4029423 | Hypoglycemic state in | ICD 9/ICD 10 | E08.65, I10:E13.649, I10:E08.649 | 1 dx |
| | diabetes | | | |
| 4042728 | Blood glucose | ICD 9/ICD 10 | I10:R73.09 | 1 dx |
| | abnormal | | | |
| 4048028 | Diabetic | ICD 9/ICD 10 | I10:E08.41, I10:E13.41 | 1 dx |
| | mononeuropathy | | | |
| 4095288 | Diabetic coma with | ICD 9/ICD 10 | I10:E13.11, E08.11, E09.11 | 1 dx |
| | ketoacidosis | | | |
| 4096666 | Diabetes mellitus with | ICD 9/ICD 10 | E13.01, E08.01, E13.00 | 1 dx |
| | hyperosmolar coma | | | |
| 4114427 | Diabetic neuropathic | ICD 9/ICD 10 | I10:E08.618, I10:E13.610, I10:E08.610, | 1 dx |
| | arthropathy | | I10:E13.618 | |
| 4174977 | Diabetic retinopathy | ICD 9/ICD 10 | I9:362.0, I10:E13.319, 362.0, I10:E13.311, | 1 dx |
| | | | 362.2, I10:E08.311, I9:362.2, I10:E08.319, | |
| | | | I10:E09.319 | |
| 4175440 | Diabetic autonomic | ICD 9/ICD 10 | I10:E08.43, 110:E13.43 | 1 dx |
| | neuropathy | | | |
| 4191611 | Diabetic amyotrophy | ICD 9/ICD 10 | E13.44, I10:E08.44 | 1 dx |
| 4214376 | Hyperglycemia | ICD 9/ICD 10 | E11.65, I10:R73.9, E10.65, I10:E13.65 | 1 dx |
| 4226798 | Hypoglycemic coma | ICD 9/ICD 10 | I10:E09.641, E08.641 | 1 dx |
| | in diabetes mellitus | | | |
| 4227657 | Diabetic skin ulcer | ICD 9/ICD 10 | I10:E13.622, E08.622 | 1 dx |
| 4308509 | Impaired fasting | ICD 9/ICD 10 | I9:790.21, I10:R73.01 | 1 dx |
| | glycaemia | | | |
| 4311629 | Impaired glucose | ICD 9/ICD 10 | I10:R73.02 | 1 dx |
| | tolerance | | | |
| 37018196 | Prediabetes | ICD 9/ICD 10 | R73.03 | 1 dx |
| 3037110 | Hemoglobin A1c/ | LOINC | 1558-6 | ≥5.7 |
| | Hemoglobin Total | | | |
| 3004410 | Hemoglobin A1c | LOINC | 4548-4 | ≥5.7 |
| | (Glycated) | | | |

TABLE 3
Obesity

| OMOP | | | | Criteria |
|---|---|---|---|---|
| Concept | OMOP | Code | | for |
| ID | Concept name | Type | Specific Code | Inclusion |
| 3038553 | Body Mass index | LOINC | 39156-5 | >=30 |
| 433736 | Obesity | ICD 9/ | I9:278.00, | 1 dx |
| | | ICD 10 | I10:E66.9, | |
| | | | I10:E66.09, | |
| | | | I10:E66.8 | |
| 434005 | Morbid Obesity | ICD 9/ | I10:E66.01, | 1 dx |
| | | ICD 10 | I9:278.01 | |
| 4060985 | Body mass index | ICD 9/ | V85.38, V85.39, | 1 dx |
| | 30+ obesity | ICD 10 | V85.41, Z68.31, | |
| | | | Z68.32, Z68.37, | |
| | | | Z68.34, Z68.35, | |
| | | | Z68.36, Z68.39 | |
| 40481140 | Childhood obesity | ICD 9/ | I9:V85.54 | 1 dx |
| | | ICD 10 | | |
| 4100857 | Extreme obesity | ICD 9/ | I9:278.03, | 1 dx |
| | with alveolar | ICD 10 | I10:E66.2 | |
| | hypoventilation | | | |
| 437525 | Overweight | ICD 9/ | E66.3 | 1 dx |
| | | ICD 10 | | |
| 4256640 | Body mass index | ICD 9/ | Z68.41, V8541 | 1 dx |
| | 40+ - severely | ICD 10 | | |
| | obese | | | |
| 4097996 | Drug-induced | ICD 9/ | E66.1 | 1 dx |
| | obesity | ICD 10 | | |

TABLE 4
Abnormal Liver Enzymes

| OMOP | | | | |
|---|---|---|---|---|
| Concept | OMOP | Code | Specific | Criteria |
| ID | Concept name | Type | Code | for Inclusion |
| 3006923 | Alanine | LOINC | 1742-6 | >=40 (2 |
| | aminotransferase | | | measurements |
| | serum/plasma | | | taken ≥ 6 |
| | | | | months apart) |
| 194984 | Disease of Liver | ICD 9/ | 573.9, 573.8, | 1 dx |
| | | ICD 10 | 572.8, | |
| | | | I10:K76.9, | |
| | | | K76.8 | |

TABLE 5
Hyperlipidemia

| OMOP | | | | Criteria |
|---|---|---|---|---|
| Concept | OMOP | Code | | for |
| ID | Concept name | Type | Specific code | Inclusion |
| 3027114 | Cholesterol | LOINC | 2093-3 | >200 |
| | [Mass/volume] | | | |
| | in Serum or Plasma | | | |
| 3035899 | Cholesterol in LDL | LOINC | 18261-8 | >=130 |
| | [Mass/volume] in | | | |
| | Serum or Plasma | | | |
| | ultracentrifugate | | | |
| 4134862 | Familial | ICD 9/ | I10:E78.01 | 1 dx |
| | hypercholesterolemia | ICD 10 | | |
| 437827 | Pure | ICD 9/ | I9:272.0, | 1 dx |
| | hypercholesterolemia | ICD 10 | I10:E78.00 | |
| 432867 | Hyperlipidemia | ICD 9/ | I9:272.4, | 1 dx |
| | | ICD 10 | I10:E78.5, | |
| | | | I10:E78.4 | |
| 438720 | Mixed | ICD 9/ | I9:272.2, | 1 dx |
| | hyperlipidemia | ICD 10 | I10:E78.2 | |

TABLE 6
Hypertension

| OMOP | | | | Criteria |
|---|---|---|---|---|
| Concept | OMOP | Code | | for |
| ID | Concept name | Type | Specific code | Inclusion |
| 320128 | Essential | ICD 9/ | I9:401.9, I10:110, | 1 dx |
| | hypertension | ICD 10 | I9:401 | |
| 312648 | Benign essential | ICD 9/ | 401.1 | 1 dx |
| | hypertension | ICD 10 | | |
| 4313767 | Chronic peripheral | ICD 9/ | 459.30, 459.31, | 1 dx |
| | venous hypertension | ICD 10 | 459.32, 459.33, | |
| | | | I9:459.39 | |
| 44782715 | Chronic peripheral | ICD 9/ | I87.312, I87.393, | 1 dx |
| | venous hypertension | ICD 10 | I87.339, I87.323, | |
| | with lower extremity | | I87.329, I87.392, | |
| | complication | | I87.391, I87.399, | |
| | | | I87.331, I87.333 | |
| 4311246 | Pre-existing | ICD 9/ | O10.013, O10.012, | 1 dx |
| | hypertension in | ICD 10 | O10.011, | |
| | obstetric context | | I10:010.019 | |
| 314958 | Benign secondary | ICD 9/ | I9:405.19,405.1 | 1 dx |
| | hypertension | ICD 10 | | |
| 312935 | Venous | ICD 9/ | I87.303, I87.302, | 1 dx |
| | hypertension | ICD 10 | I87.309, I87.301 | |
| 4064925 | Hypertension | ICD 9/ | V81.1 | 1 dx |
| | screening | ICD 10 | | |

TABLE 7
NAFLD diagnosis codes

| OMOP | | | | Criteria |
|---|---|---|---|---|
| Concept | OMOP | Code | Specific | for |
| ID | Concept Name | Type | Code | Inclusion |
| 201613 | Chronic | ICD 9/10 | I9:571.9, | 1 dx |
| | nonalcoholic | | I9:571.8 | |
| | liver disease | | | |
| 40484532 | Nonalcoholic | ICD 9/ | I10:K75.81 | 1 dx |
| | steatohopatitis | ICD 10 | | |
| | (NASH) | | | |
| 4059290 | Steatosis of liver | ICD 9/ | I10:K76.0 | 1 dx |
| | | ICD 10 | | |
| 194692 | Cirrhosis non- | ICD 9/ | I9:571.5 | 1 dx |
| | alcoholic | ICD 10 | | |
| 4064161 | Cirrhosis of liver | ICD 9/ | I10:K76.9 | 1 dx |
| | | ICD 10 | | |

Following the identification of potential NAFLD patients, patients meeting specified exclusion criteria were removed. The exclusion criteria include demonstrated alcohol use, diagnosis of HIV, viral hepatitis, type 1 diabetes, and other contributing factors that can result in hepatic steatosis or abnormal liver biochemistries. Patients on medications associated with hepatic steatosis were also excluded. All patient exclusion criteria are listed in Tables 8-13. The exclusion criteria include the followings: alcohol exclusions (Table 8), viral hepatitis exclusions (Table 9), HIV exclusions (Table 10), type 1 diabetes exclusions (Table 11), other excluding diagnoses (Table 12), and medication exclusions (Table 13). Patients meeting any one exclusion criteria were removed from the cohort. 217,969 patients were excluded from the cohort. Patients who tested with Hepatitis and/or HIV were excluded from the cohort (e.g., Positive, Reactive, Detected, Repeatedly Reactive, Confirmed, Indicated). For tests assessing viral load, patients with values above the baseline for detection were excluded.

TABLE 8
Alcohol Exclusions

| OMOP | | | | Criteria for |
|---|---|---|---|---|
| Concept Id | OMOP Concept Name | Code Type | Specific Code | Exclusion |
| 433753 | Alcohol abuse | ICD 9/ICD 10 | I9:305.00, I10:F10.10, I10:F10.129, | |
| | | | I10:F10.120, I10:F10.19, I9:305.0 | 1 dx |
| 435243 | Alcohol dependence | ICD 9/ICD 10 | I10F10.20, I10F10.21, I10F10.220 | 1 dx |
| | | | I10:F10.229, I10:F10.231, | |
| | | | I10:F10.232, I10:F10.239, | |
| | | | I10:F10.24, I9:303.90 | |
| 436953 | Continuous chronic alcoholism | ICD 9/ICD 10 | I9:303.91 | 1 dx |
| 435534 | Nondependent alcohol abuse, | ICD 9/ICD 10 | I9:305.01 | 1 dx |
| | continuous | | | |
| 375519 | Alcohol withdrawal syndrome | ICD 9/ICD 10 | I9:291.81, F10.239, I10:F10.230, | 1 dx |
| | | | F10.232 | |
| 196463 | Alcoholic cirrhosis | ICD 9/ICD 10 | K70.31, I9:571.2, I10:K70.30 | 1 dx |
| 4104431 | Alcohol intoxication | ICD 9/ICD 10 | I10:F10.120,I10:F10.129, | 1 dx |
| | | | I10:F10.920, I10:F10.929, I9:303.0 | |
| 433735 | alcoholism | ICD 9/ICD 10 | Acute alcoholic intoxication in | 1 dx |
| | | | I9:303.00, I10:F10.229, F10.220 | |
| 441276 | Nondependent alcohol abuse in | ICD 9/ICD 10 | I9:305.03 | 1 dx |
| | remission | | | |
| 201343 | Acute alcoholic liver disease | ICD 9/ICD 10 | I9:571.1, K70.10, K70.11 | 1 dx |
| 439005 | Chronic alcoholism in remission | ICD 9/ICD 10 | I10:F10.21, I9:303.93 | 1 dx |
| 377830 | Alcohol withdrawal delirium | ICD 9/ICD 10 | I9:291.0, I10:F10.231 | 1 dx |
| 437257 | Continuous acute alcoholic | ICD 9/ICD 10 | I9:303.01 | 1 dx |
| | intoxication in alcoholism | | | |
| 376383 | Alcohol-induced organic mental | ICD 9/ICD 10 | 291.8, 291.9, F10.288, F10.29, F10.9 | 1 dx |
| | disorder | | F10.94, F10.988, F10.99 | |
| 195300 | Alcoholic gastritis | ICD 9/ICD 10 | I10:K29.20, I9:535.30, I9:535.31, | 1 dx |
| | | | K29.21 | |
| 4205002 | Alcohol-induced mood disorder | ICD 9/ICD 10 | F10.14, F10.24, I10:F10.188, | 1 dx |
| | | | I10:F10.19, I10:F10.288, I10:F10.29, | |
| | | | I10:F10.94, I9:291.89 | |
| 318773 | Dilated cardiomyopathy | ICD 9/ICD 10 | I9:425.5, 142.6 | 1 dx |
| | secondary to alcohol | | | |
| 440685 | Nondependent alcohol abuse, | ICD 9/ICD 10 | I9:305.02 | 1 dx |
| | episodic | | | |
| 193256 | Alcoholic fatty liver | ICD 9/ICD 10 | I9:571.0, I10:K70.0 | 1 dx |
| 201612 | Alcoholic liver damage | ICD 9/ICD 10 | 571.3, I10:K70.9 | 1 dx |
| 378726 | Dementia associated with | ICD 9/ICD 10 | I9:291.2, I10:F10.27, I10:F10.97 | 1 dx |
| | alcoholism | | | |
| 436585 | Toxic effect of ethyl alcohol | ICD 9/ICD 10 | I9:980.0, T51.0X4A, I10:T51.0X2A, | 1 dx |
| | | | T51.0X1A | |
| 40484946 | High alcohol level in blood | ICD 9/ICD 10 | I10:Y90.0, I10:Y90.1, I10:Y90.2, | 1 dx |
| | | | I10:Y90.3, I10:Y90.4, I10:Y90.5, | |
| | | | I10:Y90.6, I10:Y90.7, I10:Y90.8 | |
| 372607 | Alcohol hallucinosis | ICD 9/ICD 10 | 291.3, F10.159, F10.251, F10.951, | 1 dx |
| | | | I10:F10.151 | |
| 374623 | Alcohol amnestic disorder | ICD 9/ICD 10 | I9:291.1, I10:F10.96, I10:F10.26 | 1 dx |
| 36714559 | Disorder caused by alcohol | ICD 9/ICD 10 | I10:F10.99, I10:F10.988 | 1 dx |
| 435532 | Episodic chronic alcoholism | ICD 9/ICD 10 | I9:303.92 | 1 dx |
| 4340383 | Alcoholic hepatitis | ICD 9/ICD 10 | I10:K70.10 | 1 dx |
| 378421 | Alcoholic polyneuropathy | ICD 9/ICD 10 | I9:357.5, I10:G62.1 | 1 dx |
| 435140 | Toxic effect of alcohol | ICD 9/ICD 10 | I9:980.9, I9:980.8, 980, | 1 dx |
| | | | I10:T51.92XA, T51.8X1A, | |
| | | | T51.8X4A, T51.94XA, T51.93XD | |
| 46269816 | Ascites due to alcoholic | ICD 9/ICD 10 | I10:K70.31 | 1 dx |
| | cirrhosis | | | |
| 441465 | Accidental poisoning by | ICD 9/ICD 10 | I9:E860.0 | 1 dx |
| | alcoholic beverage | | | |
| 4042860 | Finding relating to alcohol | SNOMED | 228273003 | 1 dx |
| | drinking behavior | | | |
| 433309 | Fetal or neonatal effect of | ICD 9/ICD 10 | I9:760.71 | 1 dx |
| | alcohol transmitted via placenta | | | |
| | and/or breast milk | | | |
| 4340493 | Alcohol-induced acute | ICD 9/ICD 10 | I10:K85.20, I10:K85.21, I10:K85.22 | 1 dx |
| | pancreatitis | | | |
| 441261 | Episodic acute alcoholic | ICD 9/ICD 10 | I9:303.02 | 1 dx |
| | intoxication in alcoholism | | | |
| 4340964 | Alcohol-induced chronic | ICD 9/ICD 10 | I10:K86.0 | 1 dx |
| | pancreatitis | | | |
| 442582 | Alcohol-induced psychotic | ICD 9/ICD 10 | I9:291.5, F10.150, I10:F10.250, | 1 dx |
| | disorder with delusions | | I10:F10.950 | |
| 436607 | Accidental poisoning by alcohol | ICD 9/ICD 10 | E860.9, I10:T51.91XA, T51.91XD, | 1 dx |
| | I9:E860.8 | | | |
| 4340386 | Alcoholic hepatic failure | ICD 9/ICD 10 | I10:K70.40, K70.41 | 1 dx |
| 435983 | Accidental poisoning with ethyl | ICD 9/ICD 10 | I9:E860.1, I10:T51.0X1A, | 1 dx |
| | alcohol | | I10:T51.0X1D | |
| 46269835 | Hepatic ascites due to chronic | ICD 9/ICD 10 | I10:K70.11 | 1 dx |
| | alcoholic hepatitis | | | |
| 4052945 | Stopped drinking alcohol | SNOMED | 4052946 | 1 dx |
| 440892 | Toxic effect of isopropyl | ICD 9/ICD 10 | I9:980.2, I10:T51.2X4A, | 1 dx |
| | alcohol | | I10:T51.2X2A | |
| 4088373 | Alcohol intoxication delirium | ICD 9/ICD 10 | F10.121, I10:F10.221, F10.921 | 1 dx |
| 432609 | Acute alcoholic intoxication in | ICD 9/ICD 10 | I9:303.03 | 1 dx |
| | remission, in alcoholism | | | |
| 4330794 | Alcohol intake exceeds | ICD 9/ICD 10 | I9:790.3 | 1 dx |
| | recommended daily limit | | | |
| 4146660 | Alcohol-induced anxiety | ICD 9/ICD 10 | F10.280, F10.980, I10:F10.180 | 1 dx |
| | disorder | | | |
| 45757093 | Alcohol dependence in | ICD 9/ICD 10 | I10:O99.310, I10:O99.311, | 1 dx |
| | pregnancy | | I10:O99.312, I10:O99.313 | |
| 4166129 | Finding of alcohol in blood | ICD 9/ICD 10 | Z02.83, I10:R78.0 | 1 dx |
| 375794 | Alcohol-induced sleep disorder | ICD 9/ICD 10 | I9:291.82, F10.982, I10:F10.282 | 1 dx |
| 4004785 | Fetal alcohol syndrome | ICD 9/ICD 10 | Q86.0 | 1 dx |
| 374317 | Alcohol-induced psychosis | ICD 9/ICD 10 | I10:F10.959, I10:F10.259, | 1 dx |
| | | | I10:F10.159 | |
| 440010 | Accidental poisoning by | ICD 9/ICD 10 | I9:E860.3, I10:T51.2X1A | 1 dx |
| | isopropyl alcohol | | | |
| 1326497 | Alcohol abuse, in remission | ICD 9/ICD 10 | I10:F10.11 | 1 dx |
| 45757783 | Gastric hemorrhage due to | ICD 9/ICD 10 | I10:K29.21 | 1 dx |
| | alcoholic gastritis | | | |
| 441761 | Methyl alcohol causing toxic | ICD 9/ICD 10 | I9:980.1 | 1 dx |
| | effect | | | |
| 37016176 | Cerebral degeneration due to | ICD 9/ICD 10 | I10:G31.2 | 1 dx |
| | alcoholism | | | |
| 46269818 | Hepatic coma due to alcoholic | ICD 9/ICD 10 | I10:K70.41 | 1 dx |
| | liver failure | | | |
| 434217 | Poisoning by alcohol deterrent | ICD 9/ICD 10 | I9:977.3, I9:E947.3 | 1 dx |
| 4176653 | Alcoholic cerebellar | ICD 9/ICD 10 | G31.2 | 1 dx |
| | degeneration | | | |
| 439277 | Alcohol withdrawal hallucinosis | ICD 9/ICD 10 | I10:F10.232 | 1 dx |
| 4078688 | Alcohol myopathy | ICD 9/ICD 10 | I10:G72.1 | 1 dx |
| 4062656 | Alcohol consumption screening | ICD 9/ICD 10 | V79.1 | 1 dx |
| 4005284 | Fetal or neonatal effect of | ICD 9/ICD 10 | I10:P04.3 | 1 dx |
| | maternal use of alcohol | | | |
| 436300 | Accidental poisoning by methyl | ICD 9/ICD 10 | E860.2 | 1 dx |
| | alcohol | | | |
| 4340385 | Alcoholic fibrosis and sclerosis | ICD 9/ICD 10 | I10:K70.2 | 1 dx |
| | of liver | | | |
| 45757131 | Alcohol dependence in | ICD 9/ICD 10 | I10:099.314 | 1 dx |
| | childbirth | | | |
| 4052946 | Alcohol consumption unknown | SNOMED | 160580001 | 1 dx |
| 4052028 | Alcohol intake within | SNOMED | 160593006 | 1 dx |
| | recommended sensible limits | | | |
| 4064179 | Maternal care for (suspected) | ICD 9/ICD 10 | O35.4XX0 | 1 dx |
| | damage to fetus from alcohol | | | |
| 4028805 | Alcohol-induced pseudo- | ICD 9/ICD 10 | I10:E24.4 | 1 dx |
| | Cushing's syndrome | | | |

TABLE 9
Viral Hepatitis Exclusions

| OMOP | | Code | Specific |
|---|---|---|---|
| concept id | OMOP Concept Name | Type | Code |
| 3002222 | Hepatitis E virus IgM Ab [Presence] in Serum | LOINC | 14212-5 |
| 3002653 | Hepatitis C virus genotype [Identifier] in Serum or Plasma by Probe | LOINC | 32286-7 |
| | and target amplification method | | |
| 3003867 | Hepatitis E virus IgG Ab [Presence] in Serum | LOINC | 14211-7 |
| 3004347 | Hepatitis D virus Ab [Presence] in Serum | LOINC | 13248-0 |
| 3008075 | Hepatitis C virus RNA [Presence] in Blood by Probe and target | LOINC | 5010-4 |
| | amplification method | | |
| 3013801 | Hepatitis C virus Ab [Presence] in Serum or Plasma by Immunoassay | LOINC | 13955-0 |
| 3014700 | Hepatitis B virus DNA [Units/volume] in Serum | LOINC | 11258-1 |
| 3016770 | Hepatitis C virus RNA [#/volume] (viral load) in Serum or Plasma | LOINC | 20416-4 |
| | by Probe and target amplification method | | |
| 3017143 | Hepatitis C virus Ab [Presence] in Serum | LOINC | 16128-1 |
| 3018447 | Hepatitis C virus RNA [Units/volume] (viral load) in Serum or | LOINC | 11011-4 |
| | Plasma by Probe and target amplification method | | |
| 3018806 | Hepatitis B virus core IgM Ab [Units/volume] in Serum | LOINC | 22319-8 |
| 3019284 | Hepatitis B virus surface Ag [Presence] in Serum | LOINC | 5195-3 |
| 3019510 | Hepatitis B virus surface Ag [Presence] in Serum or Plasma by | LOINC | 5196-1 |
| | Immunoassay | | |
| 3020316 | Hepatitis A virus IgM Ab [Presence] in Serum or Plasma by | LOINC | 13950-1 |
| | Immunoassay | | |
| 3020978 | Hepatitis B virus genotype [Identifier] in Serum or Plasma by Probe | LOINC | 32366-7 |
| | and target amplification method | | |
| 3021125 | Hepatitis C virus RNA [Presence] in Serum or Plasma by Probe and | LOINC | 11259-9 |
| | target amplification method | | |
| 3022058 | Hepatitis B virus DNA [Presence] in Serum or Plasma by Probe and | LOINC | 29610-3 |
| | target amplification method | | |
| 3022169 | Hepatitis D virus Ab [Units/volume] in Serum by Immunoassay | LOINC | 5200-1 |
| 3022560 | Hepatitis B virus core IgM Ab [Presence] in Serum or Plasma by | LOINC | 24113-3 |
| | Immunoassay | | |
| 3022900 | Hepatitis B virus polymerase DNA [Presence] in Blood by Probe and | LOINC | 16934-2 |
| | target amplification method | | |
| 3023378 | Hepatitis B virus e Ag [Presence] in Serum or Plasma by | LOINC | 13954-3 |
| | Immunoassay | | |
| 3024429 | Hepatitis C virus RNA [Units/volume] (viral load) in Serum or | LOINC | 10676-5 |
| | Plasma by Probe with amplification | | |
| 3025267 | Hepatitis B virus surface Ag [Presence] in Serum or Plasma by | LOINC | 7905-3 |
| | Neutralization test | | |
| 3026432 | Hepatitis C virus RNA [Units/volume] (viral load) in Serum or | LOINC | 29609-5 |
| | Plasma by Probe and signal amplification method | | |
| 3027346 | Hepatitis B virus DNA [#/volume] (viral load) in Serum or Plasma | LOINC | 29615-2 |
| | by Probe and target amplification method | | |
| 3030378 | Hepatitis B virus precore TAG [Presence] in Serum by Probe and | LOINC | 33633-9 |
| | target amplification method | | |
| 3032567 | Hepatitis B virus DNA [Units/volume] (viral load) in Serum or | LOINC | 42595-9 |
| | Plasma by Probe and target amplification method | | |
| 3032823 | Hepatitis C virus RNA [log units/volume] (viral load) in Serum or | LOINC | 42617-1 |
| | Plasma by Probe and signal amplification method | | |
| 3034868 | Hepatitis C virus RNA [log units/volume] (viral load) in Serum or | LOINC | 38180-6 |
| | Plasma by Probe and target amplification method | | |
| 3036806 | Hepatitis B virus e Ab [Presence] in Serum or Plasma by | LOINC | 13953-5 |
| | Immunoassay | | |
| 3038726 | Hepatitis D virus Ab [Presence] in Serum by Immunoassay | LOINC | 40727-0 |
| 3044784 | Hepatitis B Virus YMDD [Presence] in Serum or Plasma by Probe | LOINC | 43279-9 |
| | and target amplification method | | |
| 3047011 | Hepatitis D virus Ag [Presence] in Serum by Immunoassay | LOINC | 44754-0 |
| 3048505 | Hepatitis B virus DNA [log units/volume] (viral load) in Serum or | LOINC | 48398-2 |
| | Plasma by Probe and target amplification method | | |
| 3049213 | Hepatitis C virus RNA [Presence] in Unspecified specimen by Probe | LOINC | 48576-3 |
| | and signal amplification method | | |
| 3049680 | Hepatitis C virus RNA [Log #/volume] (viral load) in Serum or | LOINC | 47252-2 |
| | Plasma by Probe and target amplification method | | |
| 3052023 | Hepatitis C virus Ab Signal/Cutoff in Serum or Plasma by | LOINC | 48159-8 |
| | Immunoassay | | |
| 3053003 | Hepatitis C virus genotype [Identifier] in Blood by Probe and target | LOINC | 48574-8 |
| | amplification method | | |
| 40757341 | Hepatitis B virus basal core promoter mutation [Identifier] in Serum | LOINC | 54210-0 |
| | by Probe and target amplification method | | |
| 40759633 | Hepatitis E virus IgG Ab [Units/volume] in Serum or Plasma by | LOINC | 56513-5 |
| | Immunoassay | | |
| 40761553 | Hepatitis B virus surface Ag [Units/volume] in Serum | LOINC | 58452-4 |
| 43533679 | Hepatitis C virus NS3 gene mutations detected [Identifier] by | LOINC | 73654-6 |
| | Genotype method | | |
| 43533680 | Hepatitis C virus NS5 gene mutations detected [Identifier] by | LOINC | 73655-3 |
| | Genotype method | | |
| 43534035 | Hepatitis C virus resistance panel by Genotype method | LOINC | 72862-6 |

TABLE 10
HIV Exclusion Criteria

| OMOP | | Code | Specific |
|---|---|---|---|
| Concept Id | OMOP Concept Name | Type | Code |
| 3000685 | HIV 1 RNA [Presence] in Serum or Plasma by Probe and target | LOINC | 25835-0 |
| | amplification method | | |
| 3004365 | HIV 1 proviral DNA [Presence] in Blood by Probe with amplification | LOINC | 9837-6 |
| 3010074 | HIV 1 RNA [Log #/volume] (viral load) in Plasma by Probe and signal | LOINC | 29539-4 |
| | amplification method | | |
| HIV 1 | RNA [#/volume] (viral load) in Serum or Plasma by Probe and | | |
| 3010747 | target amplification method | LOINC | 20447-9 |
| 3011325 | HIV 1 + 2 Ab [Presence] in Serum | LOINC | 7918-6 |
| 3012693 | HIV reverse transcriptase gene mutations detected [Identifier] | LOINC | 30554-0 |
| 3012733 | HIV 2 Ab [Units/volume] in Serum or Plasma by Immunoassay | LOINC | 5224-1 |
| 3013906 | HIV 1 Ab [Presence] in Serum | LOINC | 7917-8 |
| 3014347 | HIV 1 RNA [#/volume] in Serum | LOINC | 21333-0 |
| 3016870 | HIV 1 Ab band pattern [Interpretation] in Serum by Immunoblot | LOINC | 13499-9 |
| 3017675 | HIV 1 Ab [Presence] in Serum or Plasma by Immunoassay | LOINC | 29893-5 |
| 3024449 | HIV 2 Ab [Presence] in Serum or Plasma by Immunoassay | LOINC | 30361-0 |
| 3026532 | HIV 1 RNA [Log #/volume] (viral load) in Plasma by Probe and target | LOINC | 29541-0 |
| | amplification method | | |
| 3031527 | HIV 1 RNA [#/volume] (viral load) in Serum or Plasma by Probe with | LOINC | 41515-8 |
| | amplification detection limit = 75 copies/mL | | |
| 3031839 | HIV 1 RNA [Log #/volume] (viral load) in Serum or Plasma by Probe | LOINC | 41516-6 |
| | with amplification detection limit = 1.9 log copies/mL | | |
| 3032728 | HIV genotype [Susceptibility] in Isolate by Genotype method Narrative | LOINC | 49573-9 |
| 3032965 | HIV 1 + 2 Ab [Presence] in Unspecified specimen by Rapid immunoassay | LOINC | 49580-4 |
| 3038100 | HIV 1 Ab [Presence] in Serum or Plasma by Immunoblot | LOINC | 5221-7 |
| 3039370 | HIV 2 Ab Signal/Cutoff in Serum or Plasma by Immunoassay | LOINC | 51786 -2 |
| 3039421 | HIV 1 RNA [Log #/volume] (viral load) in Serum or Plasma by Probe and | LOINC | 51780 -5 |
| | target amplification method detection limit = 0.5 log copies/mL | | |
| 3044830 | HIV protease gene mutations detected [Identifier] | LOINC | 33630-5 |
| 3045827 | HIV phenotype [Susceptibility] | LOINC | 45182-3 |
| 3047064 | HIV 1 proviral DNA [Presence] in Blood by Probe and target | LOINC | 44871-2 |
| | amplification method | | |
| 3049147 | HIV 1 + 0 + 2 Ab [Units/volume] in Serum or Plasma | LOINC | 48346-1 |
| 3053246 | HIV 1 + 0 + 2 Ab [Presence] in Serum or Plasma | LOINC | 48345-3 |
| 21494795 | HIV 1 and 2 Ab [Identifier] in Serum, Plasma or Blood by Rapid | LOINC | 80203-3 |
| | immunoassay | | |
| 40760007 | HIV 1 + 2 Ab + HIV1 p24 Ag [Presence] in Serum or Plasma by | LOINC | 56888-1 |
| | Immunoassay | | |
| 4276586 | Finding of HIV status | ICD 9/ | I10:R75 |
| | | ICD 10 | |

TABLE 11
Type 1 diabetes exclusions

| OMOP | | | | Criteria for |
|---|---|---|---|---|
| Concept Id | OMOP Concept Name | Code Type | Specific Codes | Exclusion |
| 443412 | Type 1 diabetes mellitus without | ICD 9/ICD 10 | I10:E10.9 | 1 dx |
| | complication | | | |
| 4096668 | Type 1 diabetes mellitus with gangrene | ICD 9/ICD 10 | I10:E10.52 | 1 dx |
| 4099214 | Type 1 diabetes mellitus with ulcer | ICD 9/ICD 10 | E10.621, E10.622 | 1 dx |
| 40484648 | Type 1 diabetes mellitus uncontrolled | ICD 9/ICD 10 | I9:250.03 | 1 dx |
| 201254 | Type 1 diabetes mellitus | ICD 9/ICD 10 | 250.01, I9:250.03 | 1 dx |
| 201531 | Type 1 diabetes mellitus with hyperosmolar | ICD 9/ICD 10 | 250.21 | 1 dx |
| | coma | | | |
| 318712 | Peripheral circulatory disorder associated | ICD 9/ICD 10 | 250.71, E10.51, 250.73, | 1 dx |
| | with type 1 diabetes mellitus | | I10:E10.59, E10.52 | |
| 373999 | Diabetic oculopathy associated with type 1 | ICD 9/ICD 10 | 250.51, I9:250.53, E10.39 | 1 dx |
| | diabetes mellitus | | | |
| 377821 | Neurological disorder associated with type 1 | ICD 9/ICD 10 | 250.61, I9:250.63, | 1 dx |
| | diabetes mellitus | | E10.40, I10:E10.49 | |
| 435216 | Disorder due to type 1 diabetes mellitus | ICD 9/ICD 10 | I9:250.91,I9:250.81, | 1 dx |
| | | | I9:250.83,I9:250.93, | |
| | | | E10.69, I10:E10.8 | |
| 443592 | Hyperosmolality due to uncontrolled type 1 | ICD 9/ICD 10 | 250.23 | 1 dx |
| | diabetes mellitus | | | |
| 4063042 | Pre-existing type 1 diabetes mellitus | ICD 9/ICD 10 | I10:024.03 | 1 dx |
| 4143857 | Amyotrophy due to type 1 diabetes mellitus | ICD 9/ICD 10 | E10.44 | 1 dx |
| 4224254 | Ketoacidotic coma in type 1 diabetes mellitus | ICD 9/ICD 10 | I10:E10.11 | 1 dx |
| 4225055 | Mononeuropathy associated with type 1 | ICD 9/ICD 10 | E10.41 | 1 dx |
| | diabetes mellitus | | | |
| 4225656 | Diabetic cataract associated with type 1 | ICD 9/ICD 10 | E10.36 | 1 dx |
| | diabetes mellitus | | | |
| 4227210 | Diabetic retinopathy associated with type 1 | ICD 9/ICD 10 | E10.319, I10:E10.311 | 1 dx |
| | diabetes mellitus | | | |
| 4152858 | Type 1 diabetes mellitus with arthropathy | ICD 9/ICD 10 | E10.618 | 1 dx |

TABLE 12
Other excluding diagnoses

| OMOP | | | Criteria for | |
|---|---|---|---|---|
| Concept Id | OMOP Concept Name | Code Type | Specific Code | Exclusion |
| 192275 | Alpha-1-antitrypsin deficiency | ICD 9/ICD 10 | I9:273.4, I10:E88.01 | 1 dx |
| 192675 | Biliary cirrhosis | ICD 9/ICD 10 | 571.6, K74.5 | 1 dx |
| 195856 | Cholangitis | ICD 9/ICD 10 | I9:576.1, K83.0 | 1 dx |
| 4055341 | Calculus of bile duct with cholangitis | ICD 9/ICD 10 | I10:K80.30, I10:K80.34, | 1 dx |
| | | | I10:K80.36, I10:K80.32 | |
| 4135822 | Primary biliary cholangitis | ICD 9/ICD 10 | I10:K74.3 | 1 dx |
| 46269831 | Cholangitis due to bile duct calculus | ICD 9/ICD 10 | K80.31, I10:K80.33, K80.37, | 1 dx |
| | with obstruction | | K80.35 | |
| 434614 | Disorder of iron metabolism | ICD 9/ICD 10 | E83.19, 275.0, 275.09, | 1 dx |
| | | | I10:E83.10 | |
| 436672 | Disorder of copper metabolism | ICD 9/ICD 10 | I9:275.1, E83.00, E83.09 | 1 dx |
| 438721 | Disorder of mineral metabolism | ICD 9/ICD 10 | I9:275.8, I9:275.9, 275, | 1 dx |
| | | | I10:E83.89, I10:E83.9, 275.8 | |
| 4148231 | Hereditary hemochromatosis | ICD 9/ICD 10 | 275.01, I10:E83.110, I9:275.01 | 1 dx |
| 4163735 | Hemochromatosis | ICD 9/ICD 10 | E83.111, 275.02, I9:275.03, | 1 dx |
| | | | I10:E83.119, I10:E83.118 | |
| 4234997 | Disorder of vein | ICD 9/ICD 10 | I10:187.8, I87.9, I9:453 | 1 dx |
| 37016193 | Hemochromatosis following repeated | ICD 9/ICD 10 | I10:E83.111 | 1 dx |
| | red blood cell transfusion | | | |
| 4031958 | Trace element excess | SNOMED | 238145001 | 1 dx |
| 4043346 | Disorder of thorax | ICD 9/ICD 10 | I10:S23.9XXA, I10:S24.8XXA, | 1 dx |
| | | | I10:S23.29XA, I10:S23.8XXA | |
| 4148231 | Hereditary hemochromatosis | ICD 9/ICD 10 | 275.01, E83.110 | 1 dx |
| 4064036 | Generalized skin eruption caused by | ICD 9/ICD 10 | L27.0 | 1 dx |
| | drug and medicament (DRESS | | | |
| | syndrome) | | | |
| 4058694 | Toxic liver disease with cholestasis | ICD 9/ICD 10 | K71.0 | 1 dx |
| 4058695 | Toxic liver disease with fibrosis and | ICD 9/ICD 10 | K71.7 | 1 dx |
| | cirrhosis of liver | | | |
| 4316372 | HELLP syndrome | ICD 9/ICD 10 | I10:O14.20, O14.22, | 1 dx |
| | | | I10:014.24, I10:014.25, | |
| | | | I10:014.23 | |
| 132685 | Severe pre-eclampsia - not delivered | ICD 9/ICD 10 | I9:642.53 | 1 dx |
| 438490 | Severe pre-eclampsia - delivered | ICD 9/ICD 10 | I9:642.51 | 1 dx |
| 433536 | Severe pre-eclampsia | ICD 9/ICD 10 | I9:642.5 | 1 dx |
| 4057976 | Severe pre-eclampsia with postnatal | ICD 9/ICD 10 | I9:642.54 | 1 dx |
| | complication | | | |
| 439077 | Severe pre-eclampsia - delivered with | ICD 9/ICD 10 | I9:642.52 | 1 dx |
| | postnatal complication | | | |
| 433536 | Severe pre-eclampsia | ICD 9/ICD 10 | I9:642.50 | 1 dx |
| 4151863 | Congenital abnormality of liver and/or | ICD 9/ICD 10 | O26.619 | 1 dx |
| | biliary tract | | | |
| 4062790 | Disease of the digestive system | ICD 9/ICD 10 | I10:026.613, I10:099.612, | 1 dx |
| | complicating pregnancy, childbirth | | I10:099.62, I10:099.63, | |
| | and/or the puerperium | | I10:099.611, I10:026.619 | |
| 4228429 | Carnitine deficiency | ICD 9/ICD 10 | I10:E71.40 | 1 dx |
| 195223 | Renal carnitine transport defect | ICD 9/ICD 10 | I9:277.82, I9:277.81, | 1 dx |
| | | | I10:E71.41 | |
| 432294 | Iatrogenic carnitine deficiency | ICD 9/ICD 10 | I9:277.83, I10:E71.43 | 1 dx |
| 4261777 | Ruvalcaba-Myhre syndrome | ICD 9/ICD 10 | I9:E71.440 | 1 dx |
| 45773066 | Secondary carnitine deficiency | ICD 9/ICD 10 | I10:E71.448 | 1 dx |
| 45763567 | Carnitine deficiency due to inborn error | ICD 9/ICD 10 | E71.42 | 1 dx |
| | of metabolism | | | |
| 436670 | Metabolic disease | ICD 9/ICD 10 | I9:277.9, I9:277.89, I9:277, | 1 dx |
| | | | I9:277.8, I10:E88.9 | |
| 81539 | Mitochondrial cytopathy | ICD 9/ICD 10 | I9:277.87 | 1 dx |
| 435233 | Disorder of fatty acid metabolism | ICD 9/ICD 10 | I9:277.85, I10:E71.39, | 1 dx |
| | | | I10:E71.318, I10:E71.30 | |
| 441268 | Disorder of peroxisomal function | ICD 9/ICD 10 | I9:277.86, I10:E71.548, | 1 dx |
| | | | I10:E71.50 | |
| 4079687 | Tumor lysis syndrome | ICD 9/ICD 10 | I10:E88.3, I9:277.88 | 1 dx |
| 4029270 | Carnitine nutritional deficiency | ICD 9/ICD 10 | I9:277.84 | 1 dx |
| 444421 | Alagille Syndrome (Congenital | ICD 9/ICD 10 | I10:Q44.7 | 1 dx |
| | malformation syndromes affecting | | | |
| | multiple systems) | | | |
| 44835070 | Alagille Syndrome (Congenital | ICD 9/ICD 10 | I9:759.89 | 1 dx |
| | malformation syndromes affecting | | | |
| | multiple systems) | | | |
| 434615 | Cystic fibrosis | ICD 9/ICD 10 | I9:277.00 | 1 dx |
| 45576477 | Cystic fibrosis | ICD 9/ICD 10 | I10:E84.9 | 1 dx |
| 35207084 | | | | |
| 435516 | Abetalipoproteinemia, LCAT | ICD 9/ICD 10 | I9:272.5, I10:E78.6 | 1 dx |
| | deficiency | | | |
| 134324 | Lipodystrophy | ICD 9/ICD 10 | I9:272.6, I10:E88.1 | 1 dx |
| 375241 | REYE'S SYNDROME | ICD 9/ICD 10 | I9:331.81, I10:G93.7 | 1 dx |
| 44828573 | Parenteral nutrition | ICD 9/ICD 10 | I10:V58.69 | 1 dx |
| 4082397 | Parenteral nutrition | ICD 9/ICD 10 | I10:Z76.0 | 1 dx |
| 45571391 | Parenteral nutrition | ICD 9/ICD 10 | I10:Z79.891 | 1 dx |
| 45537679 | Parenteral nutrition | ICD 9/ICD 10 | I10:Z79.899 | 1 dx |

TABLE 13
Medication Exclusions

| Anti-retroviral Medications | Other Medications |
|---|---|
| atazanavir | Amiodarone |
| darunavir (TMC114) | Tamoxifen |
| fosamprenavir | Methotrexate |
| indinavir | Cytoxan (cyclophosphamide) |
| Lopinavir | Valproate |
| ritonavir | |
| nelfinavir | |
| ritonavir | |
| saquinavir | |
| tipranavir | |
| Nucleoside/Nucleotide Reverse | |
| Transcriptase Inhibitors (NRTIs) | |
| abacavir | |
| didanosine (ddI) | |
| emtricitabine (FTC) | |
| lamivudine (3TC) | |
| stavudine (d4T) | |
| tenofovir DF | |
| zalcitabine (ddC) | |
| zidovudine (AZT) | |
| Non-Nucleoside Reverse | |
| Transcriptase Inhibitors (NNRTIs) | |
| delavirdine | |
| efavirenz | |
| Etravirine | |
| nevirapine | |
| enfuvirtide (T-20; fusion inhibitor) | |
| maraviroc (CCR5 antagonist) | |
| raltegravir (integrase inhibitor) | |

The application of the exclusions shown in Tables 8-13 produced a cohort of 624,822 potential NAFLD patients. Radiology and pathology reports (unstructured data) from 1980-2016 were used to verify hepatic steatosis in these patients. A regular expression entity-tagging approach was used to identify key words along with the usage context of these key terms. For example, the regular expression entity-tagging approach can start by finding similarities or patterns among textual data that can be then generalized to build regular expressions. In certain embodiments, the regular expression entity-tagging approach can start by supplying keyword patterns which can be then evaluated, transformed or modified until satisfying predefined terminology.

Table 14 lists various radiological modalities and the key words that were queried in the respective reports. Table 15 specifies the key terms used to identify hepatic steatosis from pathology reports obtained via liver biopsy. Hepatic steatosis was verified for 20,291 patients using this approach.

TABLE 14
Radiology modalities and key words used to identify hepatic steatosis

| | Computerized | Magnetic Resonance |
|---|---|---|
| Ultrasound | Tomography (CT) Scan | Imaging (MRI) |
| Echogenic (diffusely, | Hepatic attenuation | Signal intensity |
| increased, heterogeneous) | | |
| Hepatic steatosis | steatosis | Hepatic steatosis |
| Fatty liver | Fatty change | nodular |
| Coarsened echotexture | Heterogeneous | cirrhotic |
| | enhancement | |
| nodular | Cirrhosis/cirrhotic | |
| cirrhotic | Fatty infiltration | |

TABLE 15
Pathology key words used to identify hepatic
steatosis or steatohepatitis
Steatosis
Steatohepatitis
Non-alcoholic steatohepatitis (NASH)
Fatty liver
Cirrhosis
Non-alcoholic fatty liver disease (NAFLD)

To reduce EHR diagnosis code errors, quality control (QC) measures were employed requiring patients to have ≥2 risk factors or at least three occurrences of a given risk factor diagnosis. From the 20,291 patients with verified hepatic steatosis, 4,231 patients who were under the age of 18 or who failed the QC check were removed from the cohort. This produced a final yield of 16,060 NAFLD patients with 170 of these patients having a biopsy-proven diagnosis of NASH, the advanced phenotype of NAFLD. NASH was verified through histologic confirmation from liver biopsies.

Clinical outcomes can be predicted by fibrosis stages. Liver biopsies are sensitive techniques of detecting fibrosis stages but can be underutilized due to their invasive nature. To identify patients with higher risk features for clinically significant outcomes, noninvasive scoring systems were used to stratify patients by fibrosis stages. Here, to identify additional patients who can be at risk for developing advanced fibrosis due to NAFLD, three common fibrosis scoring metrics were applied on the 15,890 patients without histology. These metrics include the Fibrosis-4 (FIB-4) calculation, the AST to Platelet Ratio Index (APRI) calculation, and the NAFLD Fibrosis score. Data required for these calculations were extracted from each patient's clinical records. For each required variable, the mean of all measures within 1 year of the date of verified hepatic steatosis was used. For example, give a patient with verified hepatic steatosis on Jun. 20, 2017, the ALT value used in the scoring metric was the mean of all available ALT measures from Jun. 20, 2016 to Jun. 20, 2018. R was used to calculate fibrosis scores for each of the 15,890 patients. Patients who exhibited a score suggest of advanced fibrosis using at least two of the metrics were selected.

16,060 NAFLD patients were identified, with 285 having a biopsy-proven NASH diagnosis. Fibrosis scoring was performed on 15,890 patients without histology; 943 exhibited a score suggestive of advanced fibrosis (FIB-4>3.25, APRI>1.0, NAFLD FS>0.675) in ≥2 of the scoring metrics. Chart review of 100 random individuals verified 92 NAFLD patients as correctly identified by the algorithm, a positive predictive value of 92%.

In sum, NASH patients at highest risk for progressing to end-stage liver disease were identified with data commonly found in the EHR. This work highlights the use of the disclosed semi-automated algorithm in identifying NAFLD and NASH with clinical sensitivity.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein.

The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the methods and systems of the disclosed subject matter without departing from the spirit or scope of the disclosed subject matter. Thus, it is intended that the disclosed subject matter include modifications and variations that are within the scope of the appended claims and their equivalents.

What is claimed is:

1. A system for diagnosing nonalcoholic fatty liver disease (NAFLD)/nonalcoholic steatohepatitis (NASH) in patients comprising:
one or more processors; and
one or more computer-readable non-transitory storage media coupled to one or more of the processors and comprising instructions operable when executed by one or more of the processors to cause the system to:
select at least one patient with a risk indicator using an electronic health record (EHR) database, wherein the risk indicator is associated with NAFLD and/or NASH;
determine that the at least one patient fails to meet exclusion criteria; and
display the at least one patient in response to the determination.

2. The system of claim 1, wherein the system is further configured to verify hepatic steatosis of the at least one patient using a radiology report and/or a pathology report.

3. The system of claim 1, wherein the system is further configured to perform a quality control by excluding a patient who has less than two risk indicators or less than three occurrences of the risk indicator.

4. The system of claim 1, wherein the system is further configured to determine that the at least one patient receives a weight-loss surgery.

5. The system of claim 1, wherein the system is further configured to determine that the at least one patient has an end-stage liver-related outcome.

6. The system of claim 1, wherein the risk indicator is selected from the group consisting of demographic data, a diagnosis code, a procedure code, a laboratory measurement, a medication history, a pathology code, a radiology code, and combinations thereof.

7. The system of claim 6, wherein the diagnosis codes are selected from the group consisting of type 2 diabetes, obesity, abnormal liver enzymes, hyperlipidemia, hypertension, chronic nonalcoholic liver disease, nonalcoholic steatohepatitis, steatosis, cirrhosis, and combinations thereof.

8. The system of claim 1, wherein the exclusion criteria are selected from the group consisting of demographic data, a diagnosis code, a procedure code, a laboratory measurement, a medication history, a pathology code, a radiology code, and combinations thereof.

9. The system of claim 8, wherein the exclusion criteria comprise alcohol abuse, type 1 diabetes, viral hepatitis infection, HIV infection, age, or combinations thereof.

10. The system of claim 2, wherein the radiology report is selected from the group consisting of an ultrasound report, a CT scan report, a MRI report, and combinations thereof.

11. The system of claim 4, wherein the weight-loss surgery is selected from the group consisting of a laparoscopy procedure, a gastric restrictive procedure, a bariatric procedure, a bariatric revision, and combinations thereof.

12. The system of claim 5, wherein the end-stage liver-related outcome is selected from the group consisting of Model for End Stage Liver Disease (MELD) score, portal hypertension, hepatorenal syndrome, primary bacterial peritonitis, ascites, complications of transplanted liver, hepatic encephalopathy, cirrhosis, hepatocellular carcinoma, hepatopulmonary syndrome, hepatic failure, esophageal varices, esophagogastroduodenoscopy and combinations thereof.

13. A method for diagnosing nonalcoholic fatty liver disease (NAFLD)/nonalcoholic steatohepatitis (NASH) in patients comprising:
selecting at least one patient with a risk indicator using an electronic health record (EHR) database, wherein the risk indicator is associated with NAFLD and/or NASH;
determining that the at least one patient fails to meet exclusion criteria; and
displaying the at least one patient in response to the determination.

14. The method of claim 13, further comprising verifying hepatic steatosis of the at least one patient using a radiology report and/or a pathology report.

15. The method of claim 13, further comprising performing a quality control by excluding a patient who has less than two risk indicators or less than three occurrences of the risk indicator.

16. The method of claim 13, further comprising determining that the at least one patient receives a weight-loss surgery.

17. The method of claim 13, further comprising determining that the at least one patient has an end-stage liver-related outcome.

18. The method of claim 13, wherein the risk indicator is selected from the group consisting of type 2 diabetes, obesity, abnormal liver enzymes, hyperlipidemia, hypertension, chronic nonalcoholic liver disease, nonalcoholic steatohepatitis, steatosis, cirrhosis, and combinations thereof.

19. The method of claim 13, wherein the exclusion criteria comprise alcohol abuse, type 1 diabetes, viral hepatitis infection, HIV infection, age, or combinations thereof.

20. The method of claim 17, wherein the end-stage liver-related outcome is selected from the group consisting of MELD score, portal hypertension, hepatorenal syndrome, primary bacterial peritonitis, ascites, complications of transplanted liver, hepatic encephalopathy, cirrhosis, hepatopulmonary syndrome, hepatic failure, esophageal varices, esophagogastroduodenoscopy and combinations thereof.
